(19) United States
(12) Patent Application Publication
(10) Pub. No.: US 2024/0285152 A1
(43) Pub. Date: Aug. 29, 2024

(54) ENDOSCOPE SYSTEM, METHOD FOR CONTROLLING ENDOSCOPE SYSTEM, AND RECORDING MEDIUM

(71) Applicants: OLYMPUS CORPORATION, Tokyo (JP); National Cancer Center, Tokyo (JP)

(72) Inventors: Mingxuan DAI, Tokyo (JP); Hiroto OGIMOTO, Tokyo (JP); Hiro HASEGAWA, Tokyo (JP); Daichi KITAGUCHI, Tokyo (JP)

(73) Assignees: OLYMPUS CORPORATION, Tokyo (JP); National Cancer Center, Tokyo (JP)

(21) Appl. No.: 18/583,630

(22) Filed: Feb. 21, 2024

Related U.S. Application Data
(60) Provisional application No. 63/448,474, filed on Feb. 27, 2023.

(30) Foreign Application Priority Data
Feb. 21, 2024 (JP) 2024-024231

(51) Int. Cl. A61B 1/00

(52) U.S. Cl. CPC A61B 1/00188; A61B 1/00006; A61B 1/00009

(57) ABSTRACT

An endoscope system includes: an endoscope configured to acquire an image; and at least one processor including hardware. The at least one processor is configured to: acquire a distance from a treatment tool to target, acquire an operation state of the treatment tool, and determine a necessity for zooming in and zooming out on the basis of at least one of the distance and the operation state.

### CROSS-REFERENCE TO RELATED APPLICATIONS

This application claims the benefit of U.S. Provisional Application No. 63/448,474, filed Feb. 27, 2023, which is incorporated by reference herein in its entirety.

This application claims the priority to Japanese Patent Application No. 2024-024231, filed on Feb. 21, 2024, which is incorporated by reference herein in its entirety.

### FIELD

The present disclosure relates to an endoscope system, a method for controlling an endoscope system, and a recording medium.

### BACKGROUND ART

In endoscopic surgery, such as laparoscopic surgery, the magnification of an image desired by an operator differs depending on the operation of a treatment tool. Hence, during the operation of the same treatment tool, the image is switched between a close-up view and a distant view according to the operation of the treatment tool. The close-up view is a high-magnification view showing a narrow range of a subject, whereas the distant view is a low-magnification view showing a wide range of the subject. The views are switched between by, for example, an assistant who moves the endoscope closer to or away from the subject in accordance with instructions from the operator. Hence, the operator needs to give instructions to the assistant.

Meanwhile, endoscope systems that automatically change the distance from an endoscope to a subject during surgery have been proposed (for example, see PTLS 1 and 2). In PTL 1, the distance from the endoscope to the distal end of a treatment tool is changed according to the type of treatment tool. In PTL 2, the distance from the endoscope to tissue is changed according to the generation and disappearance of mist from cauterized tissue.

### SUMMARY

An aspect is an endoscope system includes: an endoscope configured to acquire an image; and at least one processor comprising hardware. The at least one processor is configured to: acquire a distance from a treatment tool to target, acquire an operation state of the treatment tool, and determine a necessity for zooming in and zooming out on the basis of at least one of the distance and the operation state.

Another aspect is a method for controlling an endoscope system, the method comprising: determining whether the treatment tool is in an image acquired by an endoscope of the endoscope system, acquiring a distance from a treatment tool to tissue, acquiring an operation state of the treatment tool, and determining a necessity for zooming in and zooming out on the basis of at least one of the distance and the operation state.

When the determining determines that zooming in is necessary, performing zoom-in control to enlarge a size of a target in the image displayed on a display. When the determining determines that zooming out is necessary, performing zoom-out control to reduce the size of the target in the image displayed on the display.

Another aspect is a non-transitory computer-readable recording medium storing a control program for causing a computer to perform the control method described above.

### BRIEF DESCRIPTION OF DRAWINGS

FIG. 1 illustrates an overall structure of an endoscope system according to an embodiment.

FIG. 2 is a block diagram of the endoscope system in FIG. 1.

FIG. 3A is an image with a close-up view during a first task of a stretching phase.

FIG. 3B is an image with a close-up view during a second task of the stretching phase.

FIG. 3C is an image with a distant view during a third task of the stretching phase.

FIG. 4A is a flowchart of a control method according to a first embodiment.

FIG. 4B is a flowchart illustrating an example of step SA2 in the control method in FIG. 4A.

FIG. 4C is a flowchart illustrating an example of step SA3 in the control method in FIG. 4A.

FIG. 4D is a flowchart illustrating an example of step SA5 in the control method in FIG. 4A.

FIG. 4E is a flowchart illustrating another example of step SA5 in the control method in FIG. 4A.

FIG. 5 is a diagram illustrating combinations of the distance from the treatment tool to the tissue and action information.

FIG. 6 is an image illustrating step SA54.

FIG. 7A is an image with a close-up view during a first task of a peeling phase.

FIG. 7B is an image with a distant view during a second task of the peeling phase.

FIG. 8A is a flowchart of a control method according to a second embodiment.

FIG. 8B is a flowchart illustrating an example of step SB3 in the control method in FIG. 8A.

FIG. 8C is a flowchart illustrating another example of step SB3 in the control method in FIG. 8A.

FIG. 8D is a flowchart illustrating an example of step SB5 in the control method in FIG. 8A.

### DESCRIPTION OF EMBODIMENTS

### First Embodiment

An endoscope system, a method for controlling the endoscope system, a control program, and a recording medium according to a first embodiment will be described with reference to the drawings.

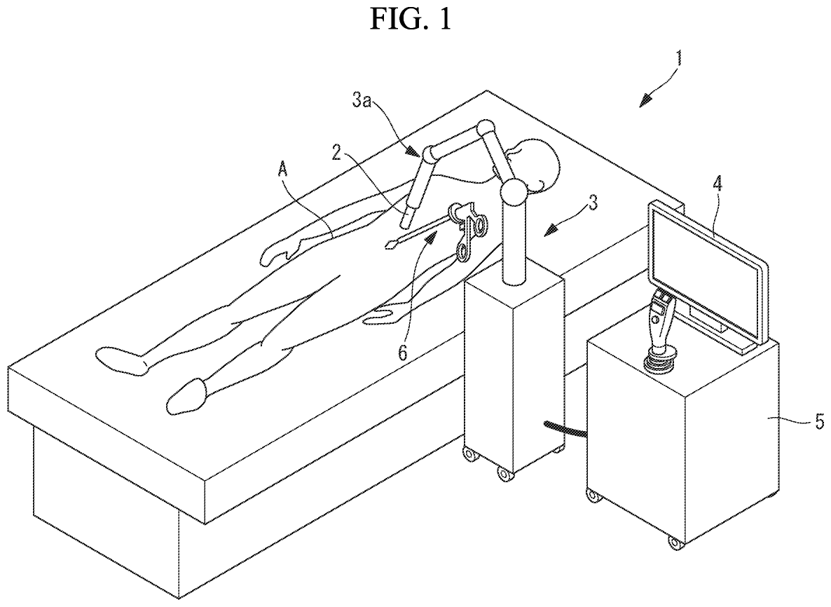

As illustrated in FIG. 1, an endoscope system 1 according to this embodiment is used for surgery, e.g., laparoscopic surgery, in which an endoscope 2 and a treatment tool 6 are inserted into the body of a patient, who is an object to be examined A, to treat a target site, such as an affected area, with the treatment tool 6 while the treatment tool 6 is observed with the endoscope 2.

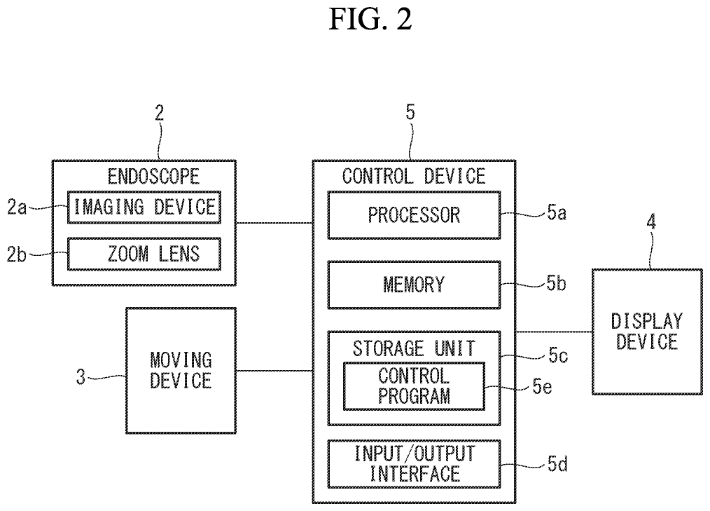

As illustrated in FIGS. 1 and 2, the endoscope system 1 includes the endoscope 2 to be inserted into the object to be examined A, a moving device 3 that changes the position and orientation of the endoscope 2, a display device 4, and a control device 5 that controls the endoscope 2 and the moving device 3.

The endoscope 2 and the treatment tool 6 are inserted into, for example, the abdominal cavity of the object to be examined A through trocars (not illustrated). The trocars are cylindrical instruments inserted into the object to be examined A through holes provided in the body wall and can be pivoted about the holes.

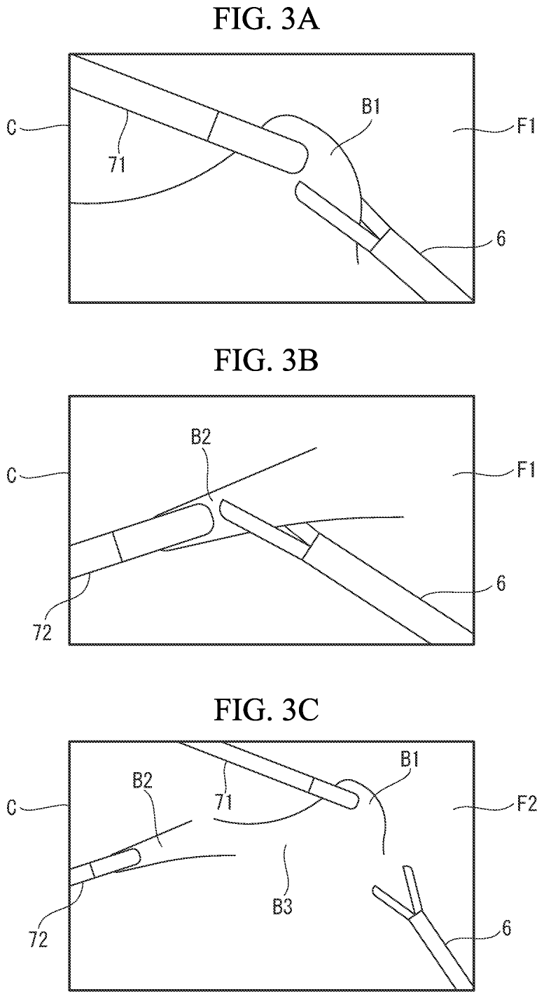

The endoscope 2 includes an imaging device (image sensor) 2a, such as a CCD image sensor or a CMOS image sensor, to acquire an image C showing tissue B (B1, B2, B3) and the treatment tool 6 (see FIGS. 3A to 3C) in the object to be examined A. The imaging device 2a is, for example, a three-dimensional camera provided at the distal end of the endoscope 2, and acquires a stereo image as the image C. An objective lens of the endoscope 2 may include a zoom lens 2b that optically enlarges or reduces a subject in the image C.

The image C is transmitted from the endoscope 2 to the display device 4 via the control device 5 and is displayed on the display device 4. The display device 4 is any type of display, such as a liquid crystal display or an organic EL display.

The moving device 3 includes an electric holder 3a, which is formed of an articulated robot arm, and is controlled by the control device 5. The endoscope 2 is held at the distal end of the electric holder 3a, and the position and orientation of the endoscope 2 are three-dimensionally changed by the operation of the electric holder 3a. The moving device 3 can include one or more joints and servo motors or other actuators providing articulation at each of the joints. The articulated robot arm can include one or more joints and corresponding actuator for articulating the one or more joints in at least 2 degrees of freedom.

The control device 5 is an endoscope processor that controls the endoscope 2, the moving device 3, and the image C displayed on the display device 4. As illustrated in FIG. 2, the control device 5 includes at least one processor 5a, a memory 5b, a storage unit 5c, and an input/output interface 5d.

The control device 5 is connected to the peripheral devices 2, 3, and 4 via the input/output interface 5d, and transmits and receives the image C, signals, and the like via the input/output interface 5d.

The storage unit 5c is a non-transitory computer-readable recording medium, and examples thereof include a hard disk drive, an optical disc, and a flash memory. The storage unit 5c stores a control program 5e for causing the processor 5a to perform a control method described below, and data necessary for the processing performed by the processor 5a.

The processor 5a performs the control method described below in accordance with the control program 5e read from the storage unit 5c into the memory 5b, such as a random access memory (RAM). A part of the processing described below performed by the processor 5a may be realized by a dedicated logic circuit, hardware, or the like, such as a field programmable gate array (FPGA), a system-on-a-chip (SoC), an application specific integrated circuit (ASIC), or a programmable logic device (PLD).

The processor 5a may control the moving device 3 in either a tracking mode or a stationary mode. The user can select one of the stationary mode and the tracking mode by using a user interface (not illustrated) provided in the control device 5.

The tracking mode is a mode in which the processor 5a controls the moving device 3 on the basis of the position of the treatment tool 6 to cause the endoscope 2 to automatically track the treatment tool 6. For example, the processor 5a acquires the position of the distal end of the treatment tool 6 by stereo measurement using the image C, and controls the moving device 3 to move the endoscope 2 such that the distal end of the treatment tool 6 is located at a predetermined target point in the image C.

The stationary mode is a mode in which the endoscope 2 is maintained at a constant position regardless of the position of the treatment tool 6.

Typically, a single surgery includes one or more treatment phases. In each treatment phase, an operator performs one type of treatment by sequentially performing multiple tasks using one treatment tool. The appropriate magnification of the subject in the image C during the surgery differs depending on the treatment phase and the task.

As illustrated in FIGS. 3A to 3C, one example of the treatment phases is a stretching phase, in which the tissue is stretched. Reference sign 6 denotes operator forceps operated by the operator, and reference signs 71 and 72 denote assistant forceps operated by an assistant.

The stretching phase includes a first task, a second task, and a third task illustrated in FIGS. 3A, 3B, and 3C, respectively.

More specifically, in the first task, the operator brings the opened operator forceps 6 close to the tissue B1, closes the operator forceps 6 to grasp the tissue B1, and delivers the tissue B1 from the operator forceps 6 to the first assistant forceps 71 (see FIG. 3A).

Next, in the second task, the operator brings the opened operator forceps 6 close to the tissue B2 at another position, closes the operator forceps 6 to grasp the tissue B2, and delivers the tissue B2 from the operator forceps 6 to the second assistant forceps 72 (see FIG. 3B).

Next, in the third task, the operator confirms that the tissue B3 between the tissue B1 and B2 is stretched by the two assistant forceps 71 and 72, and instructs the assistant to adjust the positions of the assistant forceps 71 and 72 and the directions in which the tissue B1 and B2 are pulled, as necessary (see FIG. 3C).

In the first task, the view desired by the operator is a high-magnification close-up view F1 showing the enlarged tissue B1 and treatment tools 6 and 71. In the second task, the view desired by the operator is a high-magnification close-up view F1 showing the enlarged tissue B2 and treatment tools 6 and 72. In the third task, the view desired by the operator is a low-magnification view F2 showing all of the tissue B1, B2, and B3 and the treatment tools 6, 71, and 72. FIGS. 3A and 3B illustrate the images C with the close-up view F1, and FIG. 3C illustrates the image C with the low-magnification view F2.

By performing the control method during the control of the movement device 3 in the tracking mode or the stationary mode, the processor 5a zooms in or zooms out the image C according to the treatment phase and the task, thus automatically switching between the close-up view F1 and the low-magnification view F2.

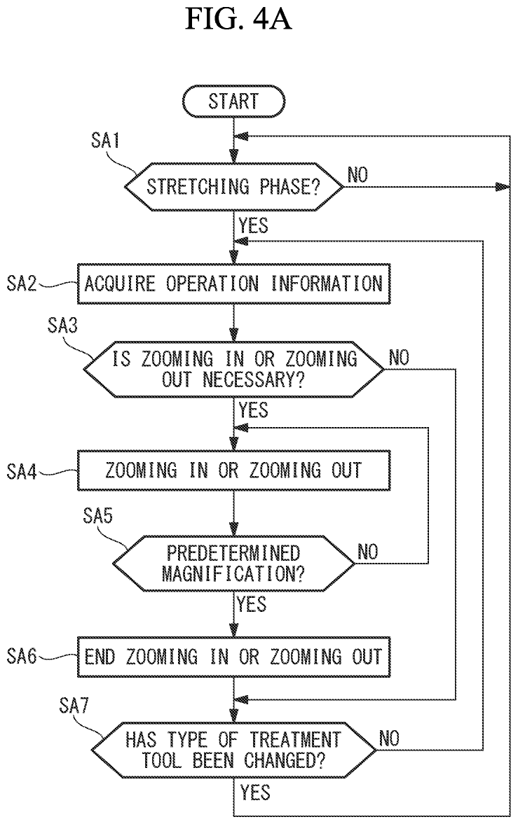

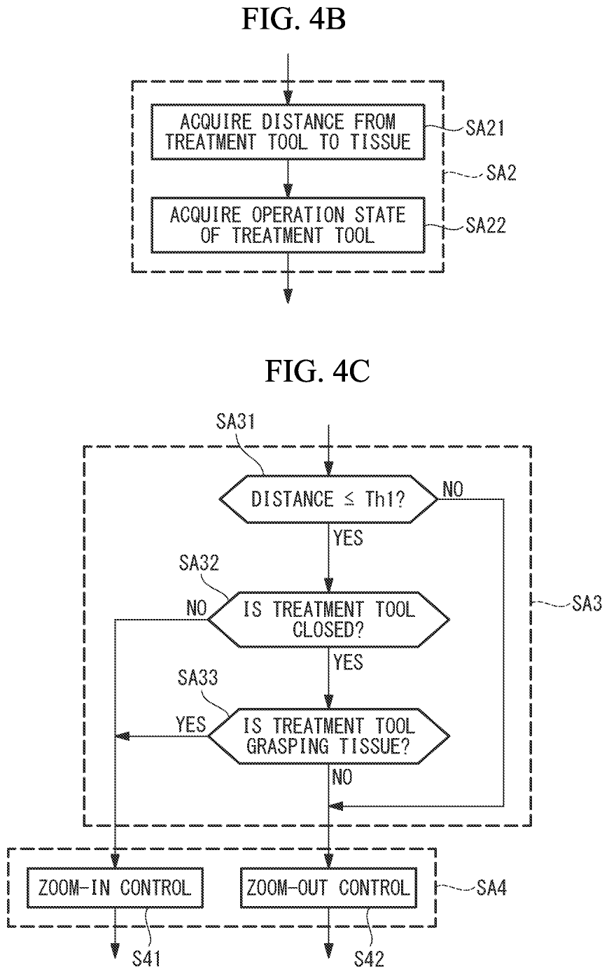

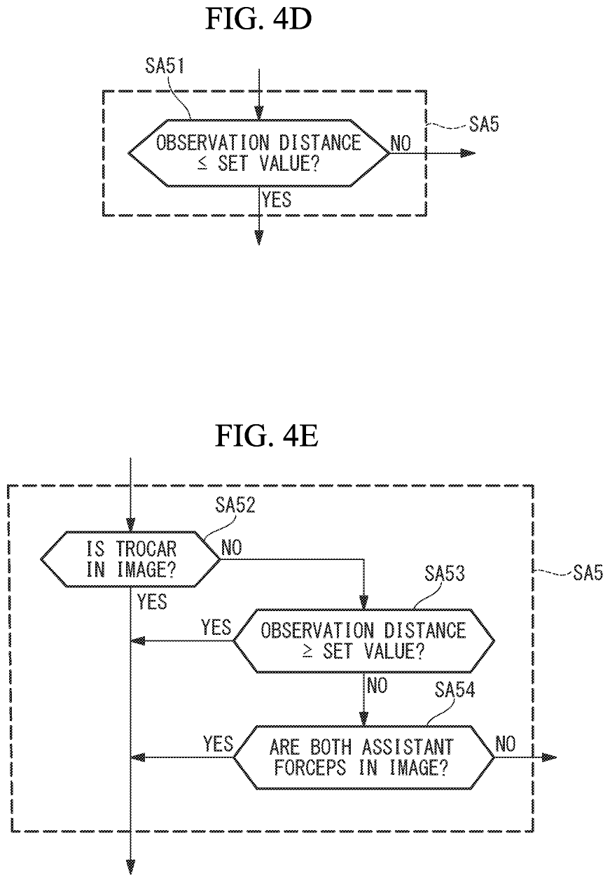

More specifically, as illustrated in FIG. 4A, the control method according to this embodiment includes step SA1 of determining the treatment phase, step SA2 of acquiring operation information about the operation of the treatment tool 6 by the operator, step SA3 of determining the necessity for zooming, step SA4 of zooming the image C, step SA5 of determining the magnification of the subject in the image C, and step SA6 of ending zooming.

The processor 5a determines the treatment phase on the basis of the image C (step SA1). More specifically, the processor 5a detects the type of the treatment tool 6 in the image C. If the treatment tool 6 is a treatment tool used for stretching, the processor 5a determines that the treatment phase is the unfolding phase (YES in step SA1), and proceeds to the next step, SA2. Meanwhile, if the treatment tool 6 is not a treatment tool used for unfolding, the processor 5a determines that the treatment phase is a phase other than the stretching phase (NO in step SA1), and repeats step SA1. Examples of the treatment tool 6 used for stretching include grasping forceps.

As illustrated in FIGS. 3A to 3C, there may be multiple treatment tools 6, 71, and 72 in the image C. Hence, in step SA1, the processor 5a may detect a predetermined target treatment tool 6 in the image C and detect the type of the target treatment tool 6. Examples of the predetermined target treatment tool 6 include a main treatment tool of the operator, a right-hand treatment tool operated by the right hand of the operator, and a treatment tool registered to the control device 5 in advance by the operator. The treatment tool is registered by, for example, setting a trocar port through which the treatment tool 6 is inserted.

The operation information includes the distance D from the treatment tool 6 to the tissue B and the operation state of the treatment tool 6. The operation state includes the open/closed state indicating whether the treatment tool 6 is open or closed, and the grasping state indicating whether the treatment tool 6 is grasping the tissue B or not. As illustrated in FIG. 4B, in step SA2, the processor 5a acquires the distance D from the treatment tool 6 to the tissue B in the depth direction of the image C (step SA21), and acquires the operation state of the treatment tool 6 (step SA22).

In step SA21, the processor 5a may acquire the distance D by measuring the distance D from the stereo image C. For example, the processor 5a measures the distance to the distal end of the treatment tool 6 in the depth direction and the distance to the background around the treatment tool 6 in the depth direction, and calculates the difference between the two distances as the distance D. The processor 5a may acquire the distance D by using another means, and, for example, may receive the distance D from a range sensor provided in the endoscope 2.

In step SA22, the processor 5a may acquire the open/closed state and the grasping state by performing image analysis of the distal portion of the treatment tool 6 in the image C. Alternatively, the processor 5a may receive, from a force sensor provided on a handle of the treatment tool 6, a force applied to the handle to determine the open/closed state and the grasping state from the magnitude of the force. The processor 5a may acquire the open/closed state and the grasping state by using other means.

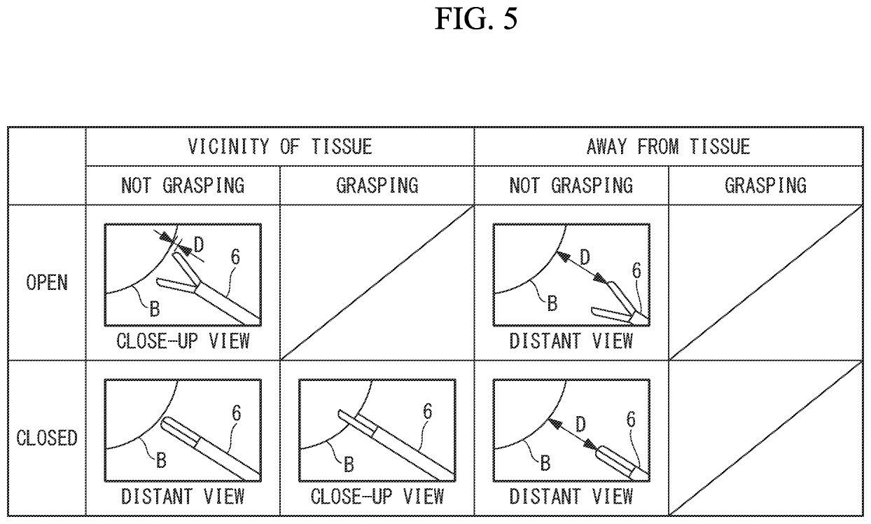

FIG. 5 illustrates combinations of the distance D and the operating state. The distance D and the operating state differ depending on the task during the stretching phase.

For example, in the first task in FIG. 3A and the second task in FIG. 3B, the grasping forceps 6 that are opened in order to grasp the tissue B are disposed near the tissue B, or the closed grasping forceps 6 are grasping the tissue B. In the third task in FIG. 3C, the grasping forceps 6 closed after releasing the tissue B are positioned in the vicinity of the tissue B, or the grasping forceps 6 are positioned at a position away from the tissue B for the next operation.

Next, the processor 5a determines the necessity for zooming in and zooming out on the basis of the distance D and the operation state in accordance with a determination flow for the stretching phase (step SA3).

FIG. 4C illustrates an example of the determination flow. The determination flow includes steps SA31 to SA33, and the processor 5a sequentially determines the distance D and the operation state in accordance with the determination flow.

More specifically, if the distance D is less than or equal to a predetermined threshold Th1 and the grasping forceps 6 are open (YES in step SA31 and NO in step SA32), or if the distance D is less than or equal to the predetermined threshold Th1 and the closed grasping forceps 6 are grasping the tissue B (YES in step SA31, YES in step SA32, and YES in step SA33), the processor 5a determines that zooming in is necessary, and performs zoom-in control for enlarging the subject in the image C displayed on the display device 4 (step S41).

In other words, the processor 5a starts zooming in by using, as a start trigger, a condition that the distance D is less than or equal to the predetermined threshold Th1 and the grasping forceps 6 are open, or a condition that the distance D is less than or equal to the predetermined threshold Th1 and the grasping forceps 6 are closed without grasping the tissue B.

Meanwhile, if the distance D is larger than the threshold Th1 (NO in step SA31), or if the grasping forceps 6 are closed without grasping the tissue B (YES in step SA31, YES in step SA32 and, NO in step SA33), the processor 5a determines that zooming out is necessary, and performs zoom-out control for reducing the subject in the image C displayed on the display device 4 (step S42).

In other words, the processor 5a starts the zoom-out control by using, as a start trigger, a condition that the distance D is larger than the threshold Th1 or a condition that the closed grasping forceps 6 are not grasping the tissue.

The predetermined threshold Th1 may be, for example, a value set in advance in the control device 5 by the operator, a value automatically set by the control device 5, or a value set by other means.

The zoom-in control and the zoom-out control are performed by controlling the position of the endoscope 2. Specifically, the processor 5a controls the moving device 3 to move the endoscope 2 closer to the subject, such as the grasping forceps 6 or the tissue, thereby zooming in the image C (step S41). The processor 5a controls the moving device 3 to move the endoscope 2 away from the subject, thereby zooming out the image C (step S42).

In steps S41 and S42, the processor 5a may perform the zoom-in control and the zoom-out control while keeping a specific point in the image C present in the image C. The specific point is, for example, a region of interest to which the operator pays attention, such as the distal end of the target treatment tool 6.

After starting to zoom in or zoom out, the processor 5a determines whether or not the subject in the image C has been zoomed in or zoomed out to a predetermined magnification (step SA5).

FIG. 4D illustrates an example method for determining the magnification in zooming in.

A predetermined first magnification in the case of zooming in is, for example, a magnification at which the observation distance equals a first set value. The observation distance is the distance from the distal end of the endoscope 2 to the subject (for example, the distal end of the treatment tool 6 or the tissue B) in the depth direction of the image C. When the observation distance becomes less than or equal to the first set value (YES in step SA51), the processor 5a stops the endoscope 2 to end the zoom-in control (step SA6).

In other words, the processor 5a ends zooming in of the image C by using, as an end trigger, a condition that the subject in the image C has been enlarged to the predetermined first magnification. As a result, the display device 4 displays the image C with the close-up view F1, in which the subject is enlarged to the predetermined first magnification, as illustrated in FIGS. 3A and 3B.

FIG. 4E illustrates an example method for determining the magnification in zooming out.

A predetermined second magnification in the case of zooming out is a magnification at which the trocar or both of the assistant forceps 71 and 72 are in the image C, or a magnification at which the observation distance equals the second set value. The second magnification is smaller than the first magnification, and the second set value is larger than the first set value. When the trocar is in the image C (YES in step SA52), when the observation distance is larger than or equal to the second set value (YES in step SA53), or when both of the assistant forceps 71 and 72 are in the image C (YES in step SA54), the processor 5a stops the endoscope 2 to end the zoom-out control (step SA6).

In other words, the processor 5a ends the zoom-out control of the image C by using, as an end trigger, a condition that the subject in the image C has been reduced to the predetermined second magnification. As a result, the display device 4 displays the image C with the low-magnification view F2, in which the subject is reduced to the predetermined second magnification, as illustrated in FIG. 3C.

The first set value and the second set value may be, for example, values set in advance in the control device 5 by the operator, values automatically set by the control device 5, or values set by other means. For example, the first set value may be a safety value to keep the distal end of the endoscope 2 from touching the subject.

In step SA52, the processor 5a may detect a trocar in the image C by using, for example, known image recognition techniques.

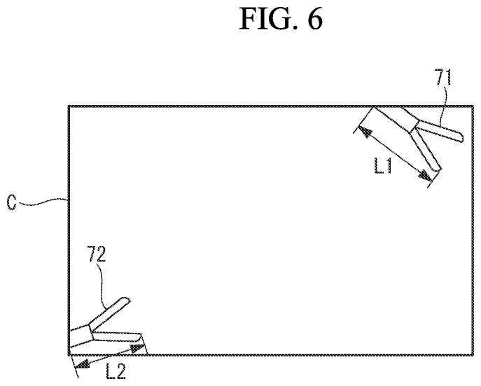

In step SA54, as illustrated in FIG. 6, the processor 5a may determine whether or not the portions of the assistant forceps 71 and 72 in the image C are larger than or equal to predetermined amounts. For example, the processor 5a may determine whether or not the lengths L1 and L2 of the portions of the assistant forceps 71 and 72 in the image C are larger than or equal to a predetermined value.

After step SA6, the processor 5a may determine whether or not the type of the treatment tool 6 has been changed (step SA7). For example, the processor 5a detects the type of the treatment tool 6 in the current image C and compares the detected type with the type of the treatment tool 6 when it is determined in step SA1 that the current treatment phase is the stretching phase, to determine if the type of the treatment tool 6 has been changed.

If the treatment tool 6 has not been changed (NO in step SA7), that is, if the stretching phase in which the same treatment tool 6 is used is continuing, the processor 5a repeats steps SA2 to SA6. Meanwhile, if the type of the treatment tool 6 has been changed (YES in step SA7), the processor 5a returns to step SA1.

As described above, according to this embodiment, the necessity for zooming in and zooming out is determined on the basis of the distance D and the operation state. The operation information, such as the distance D and the operation state, differs depending on the task during the stretching phase. Hence, it is possible to appropriately determine the necessity for zooming in and zooming out according to the task, and it is possible to automatically switch between the close-up view F1 and the low-magnification view F2 at an appropriate time according to the progress of the treatment during the stretching phase in which the same treatment tool 6 is used. This allows the operator to smoothly perform stretching of tissue.

The necessity for switching of the view according to the task differs depending on the treatment phase. According to this embodiment, it is automatically determined that the current treatment phase is the stretching phase on the basis of the image C, and then, the control method for switching the view is automatically performed. This allows the control method to be automatically performed only in the treatment phase that requires switching of the view.

Furthermore, according to this embodiment, the necessity for zooming in and zooming out is determined on the basis of the combination of the distance D, the open/closed state, and the grasping state. This makes it possible to accurately determine the necessity for zooming in and zooming out and to perform zooming in or zooming out at an appropriate time.

In this embodiment, the processor 5a determines the necessity for zooming in and zooming out on the basis of the distance D, the open/closed state, and the grasping state.

However, instead of these, the necessity may be determined on the basis of one or two of the distance D, the open/closed state, and the grasping state.

In order to appropriately determine the necessity for zooming in and zooming out, the processor 5a can perform the determination on the basis of at least two of the distance D, the open/closed state, and the grasping state.

When the determination is performed on the basis of only one of them, there may be cases where the necessity for zooming in and zooming out cannot be appropriately determined. For example, as illustrated in FIG. 5, the appropriate view when the distance D is less than or equal to the threshold Th1 can be both the close-up view F1 and the low-magnification view F2. Hence, it is difficult to appropriately determine the necessity for zooming in and zooming out on the basis of only the distance D.

### Second Embodiment

Next, an endoscope system, a method for controlling the endoscope system, a control program, and a recording medium according to a second embodiment will be described with reference to the drawings.

This embodiment differs from the first embodiment in that the processor 5a automatically performs zooming of the image C in a peeling phase, in which tissue, such as a membrane, is cut or peeled. In this embodiment, the structures different from those of the first embodiment will be described, and the structures common to those of the first embodiment will be denoted by the same reference signs and will not be described.

The endoscope system according to this embodiment has the same structure as the endoscope system 1 according to the first embodiment, and includes the endoscope 2, the moving device 3, the display device 4, and the control device 5.

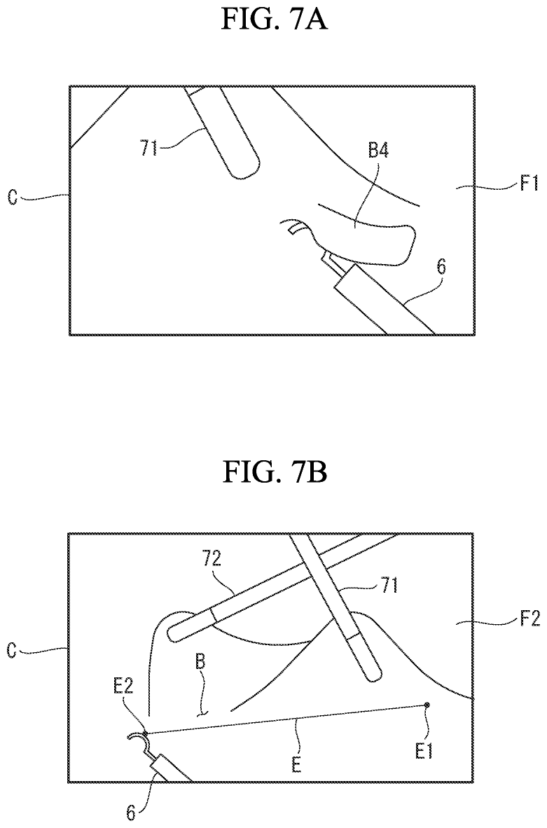

The peeling phase includes a first task and a second task, which are illustrated in FIGS. 7A and 7B, respectively.

More specifically, in the first task, the operator peels tissue B4 (for example, the intestinal membrane) with a treatment tool 6 used for peeling (see FIG. 7A). Next, in the second task, the operator observes the entire peeling line E (see FIG. 7B). Then, the operator performs additional peeling, as necessary.

In the first task, the view desired by the operator is a high-magnification close-up view F1 showing the enlarged tissue B4 and the treatment tool 6. In the second task, the view desired by the operator is a low-magnification low-magnification view F2 showing the entire peeling line E. FIG. 7A illustrates the image C with the close-up view F1, and FIG. 7B illustrates the image C with the low-magnification view F2.

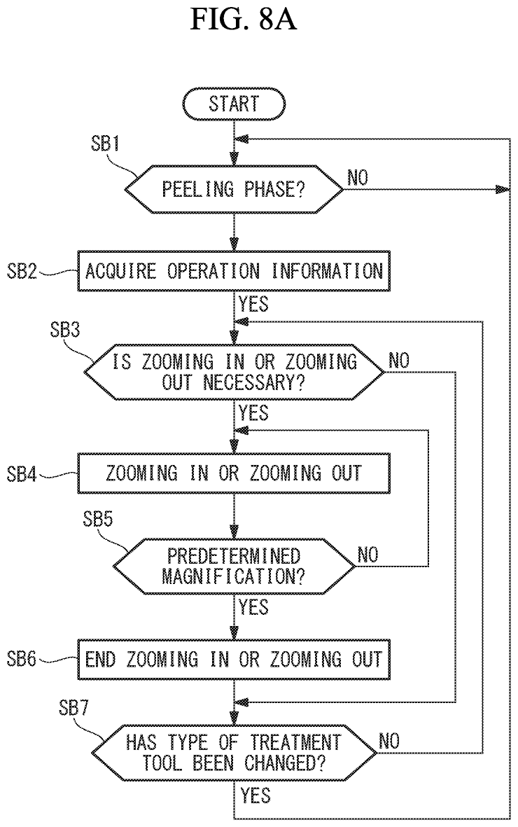

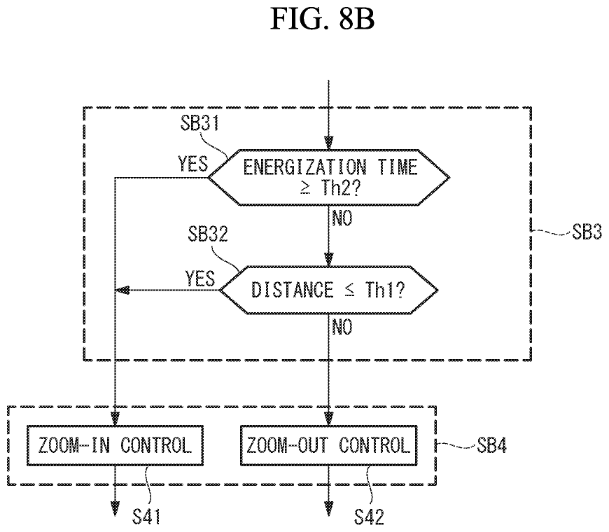

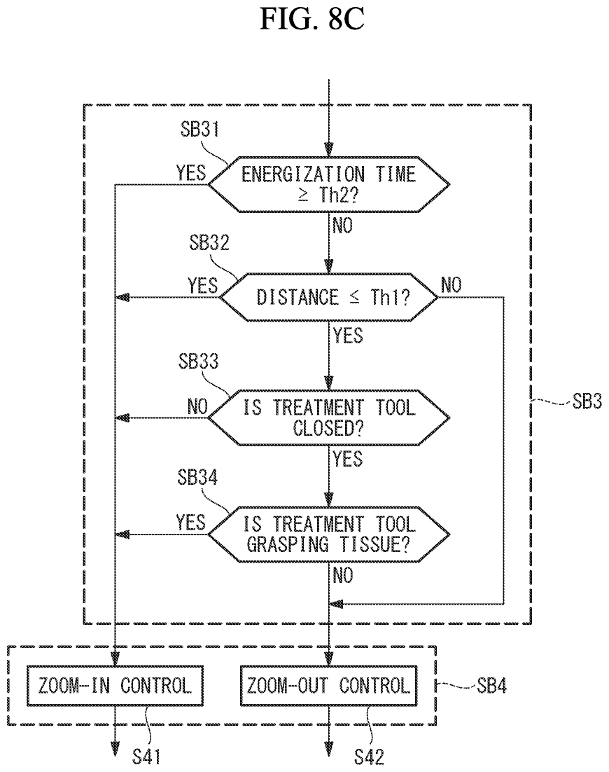

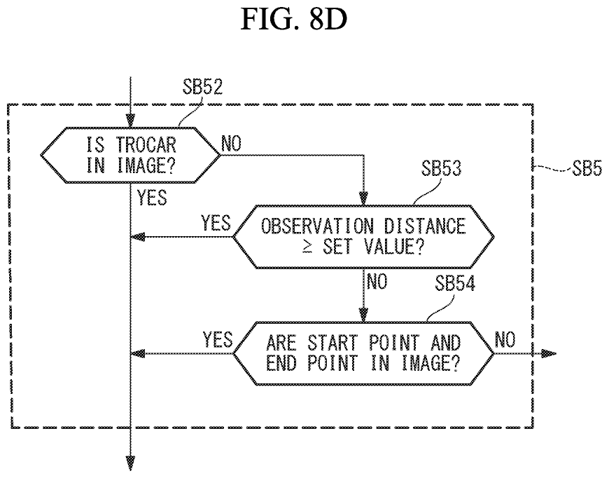

FIG. 8A illustrates a control method performed by the control device 5 in this embodiment.

The control method according to this embodiment includes step SB1 of determining the treatment phase, step SB2 of acquiring operation information about the operation of the treatment tool 6 by the operator, step SB3 of determining the necessity for zooming, step SB4 of zooming the image C, step SB5 of determining the magnification of the subject in the image C, and step SB6 of ending zooming.

The processor 5a determines the treatment phase on the basis of the image C (step SB1). More specifically, the processor 5a detects the type of the treatment tool 6 in the image C. As in step SA1, the processor 5a may detect the type of the predetermined target treatment tool 6 in the image C. If the treatment tool 6 is a treatment tool used for peeling, the processor 5a determines that the treatment phase is the peeling phase (YES in step SB1), and proceeds to the next step SB2. Meanwhile, if the treatment tool is not a treatment tool used for peeling, the processor 5a determines that the treatment phase is a phase other than the peeling phase (NO in step SB1), and repeats step SB1.

The treatment tool 6 used for peeling is, for example, either an electric scalpel or an energy device. The energy device has a pair of jaws that can be opened and closed, and supplies energy to the tissue grasped between the pair of jaws.

In step SB2, the processor 5a acquires the distance D from the treatment tool 6 to the tissue and acquires the operation state of the treatment tool 6, as in step SA2. In this embodiment, the operation state of the treatment tool 6 includes at least an energization state indicating whether or not electricity is supplied to the treatment tool 6. When the treatment tool 6 is an energy device, the operation state further includes the open/closed state indicating whether the pair of jaws are open or closed, and the grasping state indicating whether or not the pair of jaws are grasping the tissue B.

For example, the processor 5a acquires, from a generator, information about the current supplied to the treatment tool 6, and determines the energization state on the basis of the current. The generator is a device for supplying a current for peeling to the treatment tool 6 and is connected to the control device 5. The processor 5a may determine the energization state by using other means.

Next, the processor 5a determines the necessity for zooming in and zooming out on the basis of the distance D and the operation state in accordance with a determination flow for the peeling phase (step SB3).

FIG. 8B illustrates an example of the determination flow in the case where the treatment tool 6 is an electric scalpel. The determination flow includes steps SB31 and SB32, and the processor 5a sequentially determines the distance D and the operation state in accordance with the determination flow.

Specifically, if the energization time is longer than or equal to a predetermined threshold Th2 (YES in step SB31) or if the distance D is shorter than or equal to the predetermined threshold Th1 (YES in step SB32), the processor 5a determines that zooming in is necessary and performs zoom-in control (step S41). The energization time is the time elapsed since the generator starts to supply current to the treatment tool 6.

In other words, the processor 5a starts zooming in by using, as a start trigger, the condition that the energization time is longer than or equal to the predetermined threshold Th2 or the condition that the distance D is shorter than or equal to the predetermined threshold Th1.

Meanwhile, if the energization time is shorter than the threshold Th2 (NO in step SB31) or if the distance D is larger than the threshold Th1 (NO in step SB32), the processor 5a determines that zooming out is necessary and performs zoom-out control (step S42).

In other words, the processor 5a starts the zoom-out control by using, as a start trigger, the condition that the energization time is longer than or equal to the threshold Th2 or the condition that the distance D is longer than the threshold Th1.

The predetermined threshold Th2 may be, for example, a value set in advance in the control device 5 by the operator, a value automatically set by the control device 5, or a value set by other means.

FIG. 8C illustrates an example of the determination flow in the case where the treatment tool 6 is an energy device.

This determination flow further includes steps SB33 and SB34 in addition to steps SB31 and SB32. Specifically, if the distance D is less than or equal to the predetermined threshold Th1 and the energy device 6 is open (YES in step SB32 and NO in step SB33), or if the distance D is less than or equal to the predetermined threshold Th1 and the closed grasping forceps 6 are grasping the tissue (YES in step SB32, YES in step SB33, and YES in step SB34), the processor 5a determines that zooming in is necessary and performs zoom-in control (step S41).

Meanwhile, if the distance D is larger than the threshold Th1 (NO in step SB31), or if the energy device 6 is closed without grasping the tissue (YES in step SB32, YES in step SB33, and NO in step SB34), the processor 5a determines that zooming out is necessary, and performs zoom-out control (step S42).

After starting to zoom in or zoom out, the processor 5a determines whether or not the subject in the image C has been zoomed in or zoomed out to a predetermined magnification (step SB5).

A predetermined first magnification in the case of zooming in is, for example, a magnification at which the observation distance equals a first set value. As in step SA51, the processor 5a stops the endoscope 2 to end the zoom-in control when the observation distance becomes less than or equal to the first set value (step SB6).

In other words, the processor 5a ends zooming in of the image C by using, as an end trigger, the condition that the subject in the image C has been enlarged to the predetermined first magnification. As a result, the display device 4 displays the image C with the close-up view F1, in which the subject is enlarged to the predetermined first magnification, as illustrated in FIG. 7A.

FIG. 8D illustrates an example method for determining the magnification in zooming out.

The predetermined second magnification in the case of zooming out is a magnification at which the trocar or both the start point E1 and the end point E2 of the peeling line E are in the image C, or a magnification at which the observation distance equals the second set value. The second magnification is smaller than the first magnification, and the second set value is larger than the first set value. When the trocar is in the image C (YES in step SB52), when the observation distance becomes greater than or equal to the second set value (YES in step SB53), or when both the start point E1 and the end point E2 are in the image C (YES in step SB54), the processor 5a stops the endoscope 2 to end the zoom-out control (step SB6).

In other words, the processor 5a ends zooming out of the image C by using, as an end trigger, the condition that the subject in the image C has been reduced to the predetermined second magnification. As a result, the display device 4 displays the image C with the low-magnification view F2, in which the subject is reduced to the predetermined second magnification, as illustrated in FIG. 7B.

Steps SB52 and SB53 are identical to steps SA52 and SA53, respectively.

As illustrated in FIG. 7B, the start point E1 and the end point E2 are the positions of the distal end of the treatment tool 6 at the start and the end of peeling, respectively. The start point E1 and the end point E2 may be specified by the operator using an input device, or may be automatically set by the processor 5a. For example, the processor 5a may calculate the positions of the distal end of the treatment tool 6 at the start and the end of supply of current to the treatment tool 6 as the start point E1 and the end point E2, respectively, from the stereo image C. Alternatively, the processor 5a may set, as the start point E1, the position of the distal end of the treatment tool 6 at the time when it is determined that zooming in is necessary, and may set, as the end point E2, the position of the distal end of the treatment tool 6 at the time when it is determined that zooming out is necessary.

As in step SA7, after step SB6, the processor 5a may determine whether or not the type of the treatment tool 6 has been changed (step SB7).

As described above, according to this embodiment, the necessity for zooming in and zooming out is determined on the basis of the distance D and the operation state. The operation information, such as the distance D and the operation state, differs depending on the task during the peeling phase, and the current task can be recognized from the operation information. Hence, it is possible to appropriately determine the necessity for zooming in and zooming out according to the current task, and it is possible to automatically switch between the close-up view F1 and the low-magnification view F2 at an appropriate time according to the progress of the treatment during the peeling phase in which the same treatment tool 6 is used. This allows the operator to smoothly perform peeling.

Furthermore, according to this embodiment, it is automatically determined that the current treatment phase is the peeling phase on the basis of the image C, and then, the control method for switching the view is automatically performed. This allows the control method to be automatically performed only in the treatment phase that requires switching of the view.

Furthermore, according to this embodiment, the necessity for zooming in and zooming out is determined on the basis of the combination of the distance D, the energization state, the open/closed state, and the grasping state. This makes it possible to accurately determine the necessity for zooming in and zooming out and to perform zooming in or zooming out at an appropriate time.

In this embodiment, the processor 5a may determine the necessity for zooming in and zooming out on the basis of one, two, or three of the distance D, the energization state, the open/closed state, and the grasping state. In order to appropriately determine the necessity for zooming in and zooming out, the processor 5a can perform the determination on the basis of at least two of the distance D, the energization state, the open/closed state, and the grasping state.

The first embodiment and the second embodiment described above may be implemented in combination.

Specifically, in step SAL or SB1, the processor 5a may determine whether or not the treatment phase is one of the stretching phase and the peeling phase. In this case, the processor 5a performs steps SA2 to SA7 in FIG. 4A when the treatment phase is the stretching phase, and performs steps SB2 to SB7 in FIG. 8A when the treatment phase is the peeling phase.

In the first and second embodiments, the zoom-in control and the zoom-out control are performed by controlling the position of the endoscope 2. Instead, the zoom-in control and the zoom-out control may be performed by controlling the optical magnification of the endoscope 2 or by controlling the digital magnification of the image C.

Specifically, the processor 5a may control the zoom lens 2b to change the optical magnification of the endoscope 2, thereby enlarging or reducing the subject in the image C by optical zooming. Alternatively, the processor 5a may enlarge or reduce the size of the image C to enlarge or reduce the subject in the image C displayed on the display device 4 by digital zooming. In the zoom-in control and the zoom-out control, at least two of the position of the endoscope 2, the optical magnification of the endoscope 2, and the digital magnification of the image C may be controlled at the same time.

When at least one of the optical magnification and the digital magnification is controlled, the processor 5a may determine the magnification on the basis of the area of a predetermined subject in the image C (steps SA5 and SB5). The predetermined subject is, for example, the target treatment tool 6, and the predetermined first magnification in the case of zooming in may be a magnification at which the number of pixels in the region of the treatment tool 6 in the image C equals a predetermined number.

In the first and second embodiments, the processor 5a does not necessarily have to perform steps SA1 and SB1. For example, when the treatment tool 6 operated by the operator during surgery is only a treatment tool used for stretching or only a treatment tool used for peeling, steps SA1 and SB1 may be omitted.

Although the embodiments and the modifications thereof have been described above, the scope is not limited thereto, and various modifications can be made without departing from the spirit.

For example, the control method may be performed in a treatment phase other than the stretching phase and the peeling phase. The control device 5 may automatically perform only one of the zoom-in control and the zoom-out control.

### REFERENCE SIGNS LIST

1 Endoscope system
2 Endoscope
3 Moving device
4 Display device
5 Control device
5a Processor
6 Treatment tool, target treatment tool
A object to be examined
B, B1, B2, B3 Tissue
C Image
D Distance
E Peeling line
E1 Start point
E2 End point
F1 Close-up view
F2 Low-magnification view

What is claimed is:

1. An endoscope system comprising:
an endoscope configured to acquire an image; and
at least one processor comprising hardware, the at least one processor being configured to:
acquire a distance from a treatment tool to target;
acquire an operation state of the treatment tool; and
determine a necessity for zooming in and zooming out on the basis of at least one of the distance and the operation state.

2. The endoscope system according to claim 1, the endoscope system further comprising an articulated robot arm configured to change one or more of a position and an orientation of the endoscope,
wherein:
when the determining determines that zooming in is necessary, perform zoom-in control to enlarge a size of a target in the image displayed on a display; and
when the determining determines that zooming out is necessary, perform zoom-out control to reduce the size of the target in the image displayed on the display.

3. The endoscope system according to claim 2, wherein the zoom-in control and the zoom-out control are performed by controlling at least one of the position of the endoscope, an optical magnification of the endoscope, and a digital magnification of the image.

4. The endoscope system according to claim 2, wherein the at least one processor is configured to perform the zoom-in control or the zoom-out control while keeping a specific point in the image present the image.

5. The endoscope system according to claim 1, wherein the at least one processor is further configured to:
determine a treatment phase on the basis of the image; and
determine the necessity for zooming in and zooming out by sequentially determining the distance and the operation state in accordance with the determined treatment phase.

6. The endoscope system according to claim 5, wherein the at least one processor is further configured to:
detect a type of the treatment tool in the image; and
determine the treatment phase on the basis of the type of the treatment tool.

7. The endoscope system according to claim 6, wherein the at least one processor is further configured to detect the type of a predetermined target treatment tool in the image.

8. The endoscope system according to claim 6, wherein, if the type of the treatment tool is a grasping forceps, the at least one processor is configured to determine that the treatment phase is a stretching phase.

9. The endoscope system according to claim 8, wherein the operation state includes at least one of an open/closed state and a grasping state of the grasping forceps.

10. The endoscope system according to claim 6, wherein, if the type of the treatment tool detected is one of an electric scalpel and an energy device, the at least one processor is configured to determine that the treatment phase is a peeling phase.

11. The endoscope system according to claim 10, wherein the operation state includes at least one of an energization state, an open/closed state, and a grasping state of the electric scalpel or the energy device.

12. The endoscope system according to claim 1, wherein the at least one processor is configured to determine that zooming in is necessary if the acquired distance is less than or equal to a predetermined threshold.

13. The endoscope system according to claim 2, wherein the at least one processor is configured to end the zoom-in control when a magnification of the target in the image becomes higher than or equal to a predetermined first magnification.

14. The endoscope system according to claim 13, wherein the at least one processor is configured to determine the magnification on the basis of the distance from the endoscope to the target.

15. The endoscope system according to claim 14, wherein the at least one processor is configured to determine the magnification on the basis of the area of the treatment tool in the image.

16. The endoscope system according to claim 2, wherein the at least one processor is configured to end the zoom-out control when a magnification of the target in the image becomes less than or equal to a predetermined second magnification.

17. The endoscope system according to claim 16, wherein the at least one processor is configured to determine the magnification on the basis of the distance from the endoscope to the target.

18. The endoscope system according to claim 16, wherein the at least one processor is configured to determine the magnification on the basis of the area of the target in the image.

19. A method for controlling an endoscope system, the method comprising:
determining whether the treatment tool is in an image acquired by an endoscope of the endoscope system;
acquiring a distance from a treatment tool to tissue;
acquiring an operation state of the treatment tool;
determining a necessity for zooming in and zooming out on the basis of at least one of the distance and the operation state;
when the determining determines that zooming in is necessary, performing zoom-in control to enlarge a size of a target in the image displayed on a display; and
when the determining determines that zooming out is necessary, performing zoom-out control to reduce the size of the target in the image displayed on the display.

20. A non-transitory computer-readable recording medium storing a control program for causing a computer to perform the control method according to claim 19.
